(19) United States
(12) Patent Application Publication
(10) Pub. No.: US 2025/0034072 A1
(43) Pub. Date: Jan. 30, 2025

(54) SYNTHESIS METHOD OF NATURE-IDENTICAL RASPBERRY KEYTONE

(71) Applicants: Huifang DAI, Shanghai (CN); Baolu LIU, Shanghai (CN); Bin LI, Shanghai (CN); Huali DAI, Shanghai (CN); Aoxue MEI, Shanghai (CN)

(72) Inventors: Huifang DAI, Shanghai (CN); Baolu LIU, Shanghai (CN); Bin LI, Shanghai (CN); Huali DAI, Shanghai (CN); Aoxue MEI, Shanghai (CN)

(73) Assignee: Huifang DAI, Shanghai (CN)

(21) Appl. No.: 18/779,503

(22) Filed: Jul. 22, 2024

(30) Foreign Application Priority Data
Jul. 24, 2023 (CN) 202310910073.3

(51) Int. Cl. C07C 45/61; C07C 45/64; C07C 319/02; C12P 7/28

(52) U.S. Cl. CPC C07C 45/61; C07C 45/64; C07C 319/02; C12P 7/28

(57) ABSTRACT

A method for synthesizing nature-identical raspberry ketone is provided, belonging to the technical field of pharmaceutical synthesis. The nature-identical raspberry ketone is obtained by using natural p-anisaldehyde and natural acetone as initial raw materials to allow three-step continuous reactions of basic resin-catalyzed Claisen-Schmidt condensation, enzyme-catalyzed reduction, and demethylation of sodium ethanethiolate. The synthesis method is safe and clean, and has a stable yield reaching approximately 85%, providing a basis for industrial continuous production. The synthesis method not only meets the requirements for clean production, but also saves labor costs by virtue of the continuous process, and is suitable for large-scale industrial production.

### CROSS-REFERENCE TO RELATED APPLICATION

This patent application claims the benefit and priority of Chinese Patent Application No. 202310910073.3 filed with the China National Intellectual Property Administration on Jul. 24, 2023, the disclosure of which is incorporated by reference herein in its entirety as part of the present application.

### TECHNICAL FIELD

The present disclosure relates to the technical field of drug preparation and in particular to a method for synthesizing nature-identical raspberry ketone.

### BACKGROUND

Nature-identical raspberry ketone has the same clear berry aroma as naturally extracted raspberry ketone and is easier to absorb and safer than traditional chemically synthesized raspberry ketone. In 2013, GU Yuncui et al. (Flavor Fragrance Cosmetics, 2013 (Supplement 1): 39-41) used natural p-anisaldehyde as a raw material to allow a reaction with fermented acetone to undergo aldol condensation, followed by conducting hydrogenation to obtain anisyl acetone, which was subjected to demethylation to obtain natural raspberry ketone with a natural degree of not less than 98%. In 2014, XU Dongqing et al. (Chemical Research and Application, 2014, 26, 301-305) prepared natural raspberry ketone by the same method, with a total yield of 75.75% based on p-anisaldehyde and a content of up to 98.65%. The natural raspberry ketone has been widely used in daily chemicals, food, fuel, pharmaceutical intermediates, and other fields.

However, the large amount of acid and alkali used in the above processes can easily cause equipment corrosion and environmental pollution. The heavy metals such as palladium on carbon as catalysts are expensive and have poor selectivity, resulting in low intermediate content; while hydrogen reacted at high temperatures and pressure can easily cause serious and dangerous accidents if improperly operated. There is a high cost of demethylation by reflux with hydrobromic acid, reaction conditions are demanding, and a product yield hovers at 42% to 80%.

### SUMMARY

In view of this, the present disclosure provides a microchannel method for synthesizing nature-identical raspberry ketone. The synthesis method improves the specific reaction and reaction conditions based on the three steps of primary reactions: condensation, catalytic reduction, and demethylation. The synthesis method provides continuous reaction steps that are more suitable for industrial application and is a stable, efficient, and green process.

The present disclosure provides a method for synthesizing nature-identical raspberry ketone, including the following steps:
(1) conducting Claisen-Schmidt condensation, comprising: mixing p-anisaldehyde and acetone to obtain a reaction solution, introducing the reaction solution into a reactor filled with a basic anion resin through a microchannel with a diameter of approximately 2.5 mm to allow a reaction, collecting an obtained effluent and rinsing the microchannel with a small amount of the acetone, and adding water to allow dilution until solids are completely precipitated to obtain 4-(4-methoxyphenyl)but-3-en-2-one; where the reactor filled with the basic anion resin has an inner diameter of 13 mm and a filling volume of 60 mm;
(2) conducting enzyme-catalyzed reduction, comprising: adding dimethyl sulfoxide (DMSO), glucose, 4-(4-methoxyphenyl)but-3-en-2-one, an ene-reductase, glucose dehydrogenase (GDH), and nicotinamide adenine dinucleotide (NAD+) into phosphate-buffered saline (PBS) to obtain a mixed solution, stirring the mixed solution to allow a reaction at 35° C. for 24 h, maintaining a pH value of an obtained reaction solution at 7.0 by adding a saturated sodium bicarbonate solution dropwise using a pH titrator, and determining that the reaction is completed by thin layer chromatography (TLC) detection; extracting the reaction solution three times with isopropyl acetate, combining obtained isopropyl acetate phases, washing with a saturated brine solution, drying with anhydrous magnesium sulfate, and filtering to obtain a filtrate, and subjecting the filtrate to evaporation to remove solvents to obtain a product of anisyl acetone; and
(3) conducting demethylation of sodium ethanethiolate, comprising: adding an anhydrous N,N-dimethylformamide (DMF) solution of ethanethiol into an anhydrous DMF suspension of sodium hydride under a nitrogen atmosphere, stirring for 5 min to 8 min to allow full mixing to obtain a sodium ethanethiolate solution, and then adding anisyl acetone prepared in step (2) to allow heating reflux for 3 h; adjusting an obtained reaction product to a pH value of 6, extracting twice with chloroform, combining obtained organic phases, washing twice with water, conducting liquid separation, recycling chloroform by atmospheric distillation, recycling the anisyl acetone by vacuum distillation to allow reuse, and conducting recrystallization to obtain the nature-identical raspberry ketone.

Preferably, p-anisaldehyde in the reaction solution in step (1) has a volume fraction of 1% to 30%.

Preferably, the reaction solution in step (1) has a flow rate of 1.0 ml/min to 1.5 mL/min in the microchannel.

Preferably, the basic anion resin in step (1) is a hydroxide-type strongly basic anion exchange resin.

Preferably, DMSO, glucose, 4-(4-methoxyphenyl)but-3-en-2-one, the ene-reductase, the GDH, the NAD+, and a total volume of the mixed solution are at a ratio of (0.05-0.25) mL: (20-60) mg: (170-180) mg: (0.03-0.67) mg: (0.01-0.2) mg: (0.002-0.01) mg: 2 mL in the mixed solution in step (2).

Preferably, the PBS has a pH value of 7.0 and a concentration of 0.2 mol/L.

Preferably, the stirring in step (2) is conducted at 400 rpm.

Preferably, ethanethiol in the anhydrous DMF solution of ethanethiol in step (3) has a concentration of 0.03 g/mL to 0.035 g/mL; and sodium hydride in the anhydrous DMF suspension of sodium hydride has a concentration of 0.01 g/mL to 0.015 g/mL.

Preferably, the anhydrous DMF solution of ethanethiol and the anhydrous DMF suspension of sodium hydride are at a volume ratio of 1:1.

Preferably, ethanethiol, sodium hydride and anisyl acetone in step (3) are at a mass ratio of (0.3-0.35): (0.1-0.15): 1.

Compared with the prior art, the present disclosure has the following beneficial effects:

The present disclosure provides a method for synthesizing nature-identical raspberry ketone. The nature-identical raspberry ketone is obtained by using natural p-anisaldehyde and natural acetone as initial raw materials to allow three steps of continuous reactions of basic resin-catalyzed Claisen-Schmidt condensation, enzyme-catalyzed reduction, and demethylation of sodium ethanethiolate, where the basic resin-catalyzed Claisen-Schmidt condensation adopts a microchannel technology. This synthesis method is safe and clean, and has a stable yield reaching approximately 85%, providing a basis for industrial continuous production. The synthesis method not only meets the requirements for clean production, but also saves labor costs by virtue of the continuous process, and is suitable for large-scale industrial production.

### DETAILED DESCRIPTION OF THE EMBODIMENTS

The present disclosure provides a microchannel method for synthesizing nature-identical raspberry ketone.

In a specific example of the present disclosure, the basic anion resin used is purchased from Tianjin Xinyue Huamei Environmental Protection Technology Co., Ltd. and has a model number: 201×7 (OH); and
the ene-reductase used is an ene-reductase (ERED) purchased from SyncoZymes (Shanghai) Co., Ltd., with a product number of ES-ERED-128.

The present disclosure is further described below with reference to examples.

### Example 1

A method for synthesizing nature-identical raspberry ketone included the following steps:
(1) p-anisaldehyde with a volume fraction of 10% and acetone were mixed to obtain a reaction solution, the reaction solution was introduced into a reactor (with an inner diameter of 13 mm and a filling volume of 60 mm) filled with a basic anion resin through a microchannel (a diameter of the supporting pipeline was about 2.5 mm) at a flow rate of 1.5 mL/min to allow a reaction, where the reaction was repeated until completely conducted (monitored by TLC detection); the obtained effluent was collected and the microchannel was rinsed with a small amount of the acetone, and water was added to allow dilution until a solid was completely precipitated to obtain 4-(4-methoxyphenyl)but-3-en-2-one; the product was filtered and had a yield of approximately 99.1% by calculation;
(2) 54 mg of glucose, 176 mg of (E)-4-(4-methoxyphenyl)but-3-en-2-one, 0.05 mL of cosolvent DMSO, 0.15 mg of ene-reductase dry powder, 0.08 mg of GDH, and 0.01 mg of NAD+ were added into a catalytic buffer (200 mM sodium phosphate buffer, pH=7.0) to obtain a reaction system, where the reaction system was supplemented to 2 mL by the buffer; a reaction was conducted at 30°° C. and 400 rpm for 18 h, a pH value of the reaction solution was maintained by adding a saturated sodium bicarbonate solution with a pH titrator at 7.0, and the reaction was monitored by TLC detection. The reaction solution was extracted three times with isopropyl acetate, 5 mL each time, the obtained isopropyl acetate phases were combined, washed with a saturated salt solution, dried over anhydrous magnesium sulfate, and filtered to obtain a filtrate, and the filtrate was subjected to evaporation to remove solvents to obtain 220.0 mg of product anisyl acetone (4-(4-methoxyphenyl)butan-2-one), with a yield of 98.8% and a purity of 100% (HPLC); and
(3) under a nitrogen atmosphere, 0.31 g of ethanethiol was dissolved in 10 mL of anhydrous DMF to obtain an anhydrous DMF solution of ethanethiol; 0.12 g of sodium hydride was dissolved in 10 mL of anhydrous DMF to obtain an anhydrous DMF suspension of sodium hydride; the anhydrous DMF solution of ethanethiol was added into the anhydrous DMF suspension of sodium hydride, stirred for 5 min to allow full mixing to obtain a sodium ethanethiolate solution, and then 1 g of anisyl acetone prepared in step (2) was added to allow heating reflux for 3 h; an obtained reaction product was adjusted to pH=6, extracted twice with chloroform, the obtained organic phases were combined, washed twice with water, and liquid separation was conducted. Chloroform was recycled by atmospheric distillation, anisyl acetone was recycled by vacuum distillation to allow reuse, and recrystallization was conducted to obtain 0.768 g of the nature-identical raspberry ketone, with a content of 97.93% and a yield of 83.36% (based on the anisyl acetone).

The raspberry ketone had a total yield of 83.33% (based on natural p-anisaldehyde).

### Example 2

(1) p-anisaldehyde with a volume fraction of 30% and acetone were mixed to obtain a reaction solution, the reaction solution was introduced into a reactor (with an inner diameter of approximately 13 mm and a filling volume of approximately 60 mm) filled with a basic anion resin through a microchannel (a diameter of the supporting pipeline was about 2.5 mm) at a flow rate of 1.0 mL/min to allow a reaction, where the reaction was repeated until completely conducted (monitored by TLC detection). The obtained effluent was collected and the microchannel was rinsed with a small amount of acetone, and water was added to allow dilution until a solid was completely precipitated to obtain 4-(4-methoxyphenyl)but-3-en-2-one; the product was filtered and gave a yield of approximately 93.8% by calculation;
(2) 25 mg of glucose, 176 mg of 4-(4-methoxyphenyl)but-3-en-2-one, 0.1 mL of cosolvent DMSO, 0.075 mg of ene-reductase dry powder, 0.020 mg of GDH, and 0.002 mg of NADP+were added into a catalytic buffer (200 mM sodium phosphate buffer, pH=7.5) to obtain a reaction system, where the reaction system was supplemented to 2 mL by the buffer; a reaction was conducted at 35° C. and 400 rpm for 20 h, a pH value of the reaction system was maintained by adding a saturated sodium bicarbonate solution with a pH titrator at 7.5, and the reaction was monitored by TLC detection. The reaction solution was extracted three times with isopropyl acetate, 5 mL each time, the obtained isopropyl acetate phases were combined, washed with a saturated salt solution, dried over anhydrous magnesium sulfate, and filtered to obtain a filtrate, and the filtrate was subjected to evaporation to remove solvents to obtain 214.59 mg of product anisyl acetone (4-(4-methoxyphenyl)butan-2-one), with a yield of 96.37% and a purity of 100% (HPLC); and
(3) under a nitrogen atmosphere, 0.31 g of ethanethiol was dissolved in 10 mL of anhydrous DMF to obtain an anhydrous DMF solution of ethanethiol; 0.12 g of sodium hydride was dissolved in 10 mL of anhydrous DMF to obtain an anhydrous DMF suspension of sodium hydride; the anhydrous DMF solution of ethancthiol was added into the anhydrous DMF suspension of sodium hydride, stirred for 5 min to allow full mixing to obtain a sodium ethanethiolate solution, and then 1.5g of anisyl acetone prepared in step (2) was added to allow heating reflux for 4 h; The obtained reaction product was adjusted to pH=6, extracted twice with chloroform, obtained organic phases were combined, washed twice with water, liquid separation was conducted, chloroform was recycled by atmospheric distillation, anisyl acetone was recycled by vacuum distillation to allow reuse, and recrystallization was conducted to obtain 1.167 g of nature-identical raspberry ketone, with a yield of 84.44% (based on anisyl acetone).

The raspberry ketone had a total yield of 72.83% (based on natural p-anisaldehyde).

### Comparative Example 1

(1) 6.8 g water and 0.24 g NaOH were added into a 50 mL three-necked flask and stirred to dissolve. 4.34 g of natural acetone was added. 2.24 g of p-anisaldehyde was added and stirred to allow a reaction at 20° C. to 25° C. for 30 min. 16 mL of water was added into a reaction solution to allow the reaction product anisal acetone (4-(4-methoxyphenyl)but-3-en-2-one) to be fully precipitated from the reaction solution, and then the solid product was filtered under reduced pressure and dried to obtain 2.68 g of intermediate anisal acetone, with a yield of 92.5%.
(2) 13 mL of ethanol, 2.0 g of anisal acetone, and 0.2 g of palladium-on-carbon catalyst into a 50 mL round-bottom flask. A three-way valve was connected, airtightness was checked with nitrogen, gas in the reaction device was evacuated, and then hydrogen was introduced into the device to replace hydrogen three times, and the reaction was conducted by stirring at room temperature for 14 h, and the completion of the reaction was monitored by TLC. Palladium-on-carbon was recovered by filtration, and the ethanol was spin-dried to obtain 1.80 g of anisyl acetone with a yield of 89.1%.
(3) 1 g of anisyl acetone, 0.6 mL of glacial acetic acid, and 6 mL of 48% hydrobromic acid were added into a 50 mL glass three-necked reaction bottle. The raw materials were slowly heated and stirred, and refluxed at 105° C. to 110° C. for about 9 h, and completion of the reaction was monitored by TLC. The reaction product was extracted three times with ethyl acetate and then reacted with 30% sodium hydroxide solution, and completion of the reaction was monitored by TLC. Liquid separation was conducted, and hydrochloric acid was added into an aqueous layer until the pH value was 6. The aqueous phases were extracted three times with ethyl acetate and the organic phases were combined, and then evaporated to dryness to obtain 0.837 g of product, with a purity of 78.6% and a yield of 69.7% (based on anisyl acetone).

The raspberry ketone had a total yield of 57.5% (based on natural p-anisaldehyde).

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A method synthesizing nature-identical raspberry ketone, comprising the following steps:
(1) conducting Claisen-Schmidt condensation, comprising: mixing p-anisaldehyde and acetone to obtain a reaction solution, introducing the reaction solution into a reactor filled with a basic anion resin through a microchannel with a diameter of approximately 2.5 mm to allow a reaction, collecting an obtained effluent and rinsing the microchannel with a small amount of the acetone, and adding water to allow dilution until solids are completely precipitated to obtain 4-(4-methoxyphenyl)but-3-en-2-one; wherein the reactor filled with the basic anion resin has an inner diameter of 13 mm and a filling volume of 60 mm;
(2) conducting enzyme-catalyzed reduction, comprising: adding dimethyl sulfoxide (DMSO), glucose, 4-(4-methoxyphenyl)but-3-en-2-one, an ene-reductase, glucose dehydrogenase (GDH), and nicotinamide adenine dinucleotide (NAD+) into phosphate-buffered saline (PBS) to obtain a mixed solution, stirring the mixed solution to allow a reaction at 35° C. for 24 h, maintaining a pH value of an obtained reaction solution at 7.0 by adding a saturated sodium bicarbonate solution dropwise using a pH titrator, and determining that the reaction is completed by thin layer chromatography (TLC) detection; extracting the reaction solution three times with isopropyl acetate, combining obtained isopropyl acetate phases, washing with a saturated salt solution, drying over anhydrous magnesium sulfate, and filtering to obtain a filtrate, and subjecting the filtrate to evaporation to remove solvents to obtain a product anisyl acetone; and
(3) conducting demethylation of sodium ethanethiolate, comprising: adding an anhydrous N,N-dimethylformamide (DMF) solution of ethanethiol into an anhydrous DMF suspension of sodium hydride under a nitrogen atmosphere, stirring for 5 min to 8 min to allow full mixing to obtain a sodium ethanethiolate solution, and then adding anisyl acetone prepared in step (2) to allow heating reflux for 3 h; adjusting an obtained reaction product to a pH value of 6, extracting twice with chloroform, combining obtained organic phases, washing twice with water, conducting liquid separation, recycling chloroform by atmospheric distillation, recycling anisyl acetone by vacuum distillation to allow reuse, and conducting recrystallization to obtain nature-identical raspberry ketone.

2. The method according to claim 1, wherein p-anisaldehyde in the reaction solution in step (1) has a volume fraction of 1% to 30%.

3. The method according to claim 1, wherein the reaction solution in step (1) has a flow rate of 1.0 mL/min to 1.5 mL/min in the microchannel.

4. The method according to claim 1, wherein the basic anion resin in step (1) is a hydroxide-type strongly basic anion exchange resin.

5. The method according to claim 1, wherein DMSO, glucose, 4-(4-methoxyphenyl)but-3-en-2-one, the ene-reductase, the GDH, the NAD+, and a total volume of the mixed solution are at a ratio of (0.05-0.25) mL: (20-60) mg: (170-180) mg: (0.03-0.67) mg: (0.01-0.2) mg: (0.002-0.01) mg: 2 mL in the mixed solution in step (2).

6. The method according to claim 5, wherein PBS has a pH value of 7.0 and a concentration of 0.2 mol/L.

7. The method according to claim 1, wherein the stirring in step (2) is conducted at 400 rpm.

8. The method according to claim 1, wherein ethanethiol in the anhydrous DMF solution of ethanethiol in step (3) has a concentration of 0.03 g/mL to 0.035 g/mL; and sodium hydride in the anhydrous DMF suspension of sodium hydride has a concentration of 0.01 g/mL to 0.015 g/mL.

9. The method according to claim 8, wherein the anhydrous DMF solution of ethanethiol and the anhydrous DMF suspension of sodium hydride are at a volume ratio of 1:1.

10. The method according to claim 1, wherein ethanethiol, sodium hydride and anisyl acetone in step (3) are at a mass ratio of (0.3-0.35):(0.1-0.15):1.
